(19) United States
(12) Patent Application Publication
(10) Pub. No.: US 2011/0082390 A1
(43) Pub. Date: Apr. 7, 2011

(54) COMPLIANT PRESSURE ACTUATED SURFACE SENSOR FOR ON BODY DETECTION

(76) Inventors: MARCUS KRIETER, NEWPORT BEACH, CA (US); ZEEV COLLIN, TUSTIN, CA (US)

(21) Appl. No.: 12/573,951

(22) Filed: Oct. 6, 2009

(51) Int. Cl. A61B 5/103

(52) U.S. Cl. 600/587

(57) ABSTRACT

A system for detecting actuation pressure is provided, where actuation pressure is a pressure above a pre-defined limit on the surface of a body of a user. The system includes a multi-layered pad of flexible materials which remains in contact with the surface of the body while complying with the shape of the surface of the body. The system also includes a plurality of pressure detection zones. The plurality of pressure detection zones is located on the multi-layered pad to detect actuation pressure on the surface of the body.

### FIELD OF INVENTION

The invention relates to devices for detecting pressure, and more particularly, to system and method for detecting pressure applied on a body surface.

### BACKGROUND

Diabetic induced neuropathy can cause sufferers to lose all sensation in their feet. Because of this, objects or other physical aberrations statically captured between the bottom of a sufferer's foot and the interior of the sole of their shoe can cause a persistent pressure gradient to form in the involved region. Individuals with normal sensation in their feet would feel an acute or gradually increasing sensation of pain in the area affected. This would cause them to take action, such as moving their foot or removing the object or aberration, to relieve the pain and hence the pressure. Those without the ability to feel pain in this area, however, may easily allow this pressure gradient to persist for extended periods thereby causing tissue breakdown and subsequently development of ulcerative or other degenerative conditions because of this.

Every year thousands of diabetics loose all or a portion of their feet to medical amputation because of complications due to sores they receive to the bottom of their feet. Diabetics often suffer from peripheral neuropathy of the feet as a consequence of their disease.

In recent years, there has been growing interest to understand stresses associated complications with diabetes that can lead to infection and subsequent amputation. A capacitive biofeedback sensor that uses a polyurethane dielectric sandwiched between two wire mesh or carbon impregnated silicone rubber conductors has been disclosed by U.S. Pat. No. 5,775,332 (Goldman). Means of measuring localized plantar pressure and shear with a fiber-optic sensor array has been attempted by W. C. Wang and others (“A shear and plantar pressure sensor based on fiber-optic bend loss”, J. of Re-habilitation Research & Development. 2005 June; Volume 42, Number 3, Pages 315-326).

In light of the foregoing discussion, there is a need of a simple system and a method to preventing such degenerative conditions, such as ulcers, that are a precursor to conditions requiring amputation.

### BRIEF SUMMARY OF THE INVENTION

An object of the present invention is to provide a system for detecting pressure on a body surface which could lead to tissue breakdown and subsequently development of ulcerative or other degenerative conditions.

To achieve the objects of the present invention, an embodiment of the present invention provides a system and a method for detecting actuation pressure, where actuation pressure is a pressure above a pre-defined limit on the body surface.

### BRIEF DESCRIPTION OF THE DRAWINGS

The preferred embodiments of the invention will hereinafter be described in conjunction with the appended drawings provided to illustrated and not to limit the invention, wherein like designations denote like elements, and in which:

FIG. 1 is a side view of an orthotic insert including the body surface pressure detection system that would lie on top of the interior sole of a shoe, in accordance with an embodiment of the present invention.

FIG. 2 is a bottom view of a plurality of isolated pressure monitoring zones located on the orthotic insert, in accordance with an embodiment of the present invention.

FIG. 3 is a side view of an orthotic insert illustrating the development of a pressure gradient due to intrusion of a foreign object between the body surface and the orthotic insert, in accordance with an embodiment of the present invention.

FIG. 4 is a schematic of a micro-controller unit monitoring the plurality of isolated pressure monitoring zones, in accordance with an embodiment of the present invention.

FIG. 5 is a flow diagram illustrating the method for detection of pressure on the body surface, in accordance with an embodiment of the present invention.

FIG. 6 is a diagram showing an exemplary arrangement of the components of the body surface pressure detection system with an integrated alert means, in accordance with an embodiment of the present invention.

FIG. 7 is a diagram showing an exemplary arrangement of the components of the body surface pressure detection system with a wireless alert indication means, in accordance with an embodiment of the present invention.

FIG. 8A is a side-view of a multi-layered mat of flexible materials included in the body surface pressure detection system, in accordance with an embodiment of the present invention.

FIG. 8B is a magnified side-view of a multi-layered mat of flexible materials included in the body surface pressure detection system, in accordance with an embodiment of the present invention.

FIG. 9 is a side exploded view of the multi-layered pad, in accordance with an embodiment of the present invention.

FIG. 10 is a view of a single column of a foam compression spacer with a single nub of a conductive rubber pad, in accordance with an embodiment of the present invention.

FIG. 11 is a perspective exploded view of the multi-layered pad, in accordance with an embodiment of the present invention.

FIG. 12 is a diagram illustrating the formation of electrical connections on the multi-layered pad, in accordance with an embodiment of the present invention.

FIG. 13 is a diagram illustrating the breaking of electrical connections on the multi-layered pad, in accordance with an embodiment of the present invention.

FIG. 14 is a perspective view of a cutaway of the multi-layered pad, in accordance with an embodiment of the present invention.

FIG. 15 is a side view of a multi-layered pad illustrating the placement of a lateral insulator between two electrically isolated conductive rubber contacts, in accordance with an embodiment of the present invention.

### DETAILED DESCRIPTION OF THE EMBODIMENTS

In the following detailed description of the embodiments of the invention, numerous specific details are set forth in order to provide a thorough understanding of the embodiments of the invention. However, it will be obvious to one skilled in the art that the embodiments of the invention may be practiced without these specific details. In other instances well known methods, procedures, components, and circuits have not been described in detail so as not to unnecessarily obscure aspects of the embodiments of the invention.

Furthermore, it will be clear that the invention is not limited to theses embodiments only. Numerous modifications, changes, variations, substitutions and equivalents will be apparent to those skilled in the art without parting from the spirit and scope of the invention.

The embodiments of the invention include a method, and system for detecting actuation pressure, wherein actuation pressure is a pressure above a pre-defined limit on surface of a body of a user. In this context, detecting actuation pressure sensor also includes an orthotic multilayered pad that may put in contact of the body surface to detect the pressure.

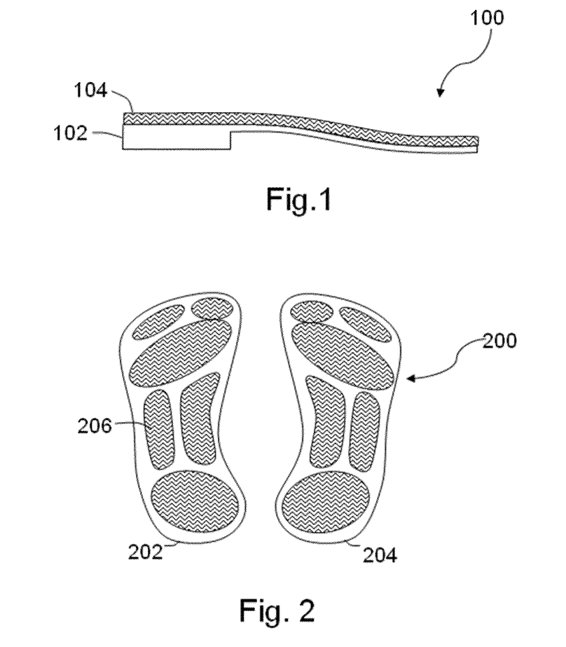

FIG. 1 is a side view of an orthotic insert including the body surface pressure detection system that would lie on top of the interior sole of a shoe, in accordance with an embodiment of the present invention. FIG. 1 illustrates an Orthotic Insert 104 including the body surface pressure detection system that would lie on top of the interior sole 102 of a shoe, in accordance with an embodiment of the present invention. The Orthotic Insert 104 would have a plurality of pressure detection zones located in areas of the foot at risk for degenerative conditions.

FIG. 2 is a bottom view of a plurality of isolated pressure monitoring zones located on the orthotic insert, in accordance with an embodiment of the present invention. FIG. 2 illustrates a plurality of pressure detection zones 206 located on the Orthotic Insert 104, in accordance with an embodiment of the present invention. Further, FIG. 2 shows that the pressure detection zones 206 are physically isolated. Moreover, the pressure detection zones 206 are electrically isolated.

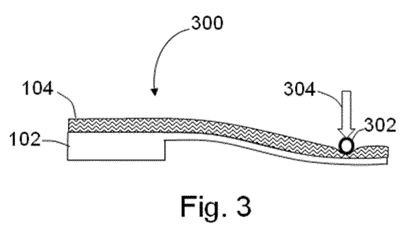

FIG. 3 is a side view of an orthotic insert illustrating the development of a pressure gradient due to intrusion of a foreign object between the body surface and the orthotic insert, in accordance with an embodiment of the present invention. FIG. 3 illustrates development of a pressure gradient due to intrusion of a foreign object between the body surface and the orthotic insert, in accordance with an embodiment of the present invention. In case of presence of a non-compliant foreign object 302 or other such arborous intrusion between the Orthotic Insert 104 and the foot and persistence of this foreign object 302 in a stationary location, a pressure 304 gradient forms between the foot and the object 302. This development of a pressure 304 gradient due to intrusion of the foreign object 302 between the body surface and the Orthotic Insert 104 is illustrated in FIG. 3. The pressure 304 gradient is transferred through the Orthotic Insert 104, causing the Orthotic Insert 104 to be deformed as shown in FIG. 3.

The body surface pressure detection system detects the deformation when the resultant pressure reaches or exceeds a pressure of sufficient magnitude to cause tissue degeneration, hereinafter referred to as actuation pressure, if allowed to persist beyond a predetermined time limit tₘₐₓ.

Actuation pressure causes an electrical change that can be detected by a computational device included in the body surface pressure detection system.

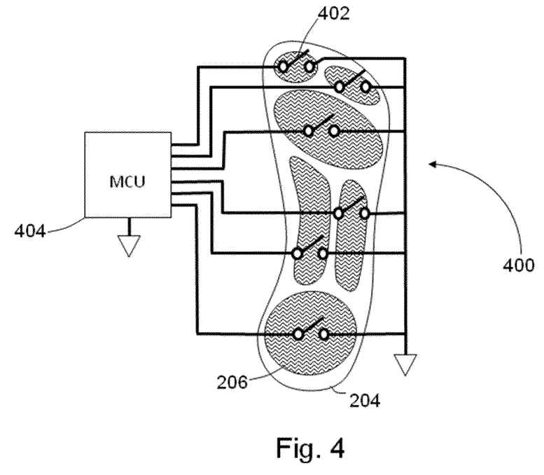

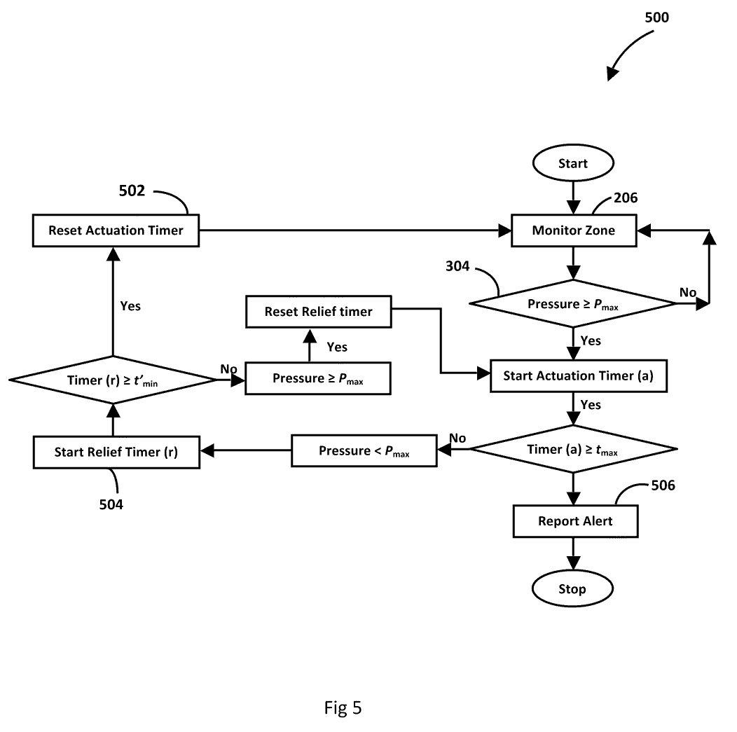

FIG. 4 is a schematic diagram of a micro-controller unit monitoring the plurality of isolated pressure monitoring zones, in accordance with an embodiment of the present invention. In an embodiment of the present invention, the computational device can be a Micro Controller Unit 404 monitoring the plurality of isolated pressure detection zones 206, as illustrated in FIG. 4. Although an MCU 404 can detect that change through numerous existing methods such as using an Analog to Digital converter (A/D) or analog comparator, the body surface pressure detection system can be constructed to allow detection using simple digital General Purpose Input Output (GPIO) lines. The Orthotic Insert 104 would be constructed to act as a series of digital switches 402; one for each isolated pressure detection zone. Past the actuation pressure, each digital switch 402 would actuate causing a change in electrical conduction sufficient to register a logical change of state with the MCU 404 on a dedicated GPIO pin.

Once a change of state is detected by the MCU 404 in a zone 206, the MCU 404 would start a time base algorithm for that zone 206 that would measure the continuous duration, hereinafter referred to as a first time period, for which the actuation pressure was maintained within the zone 206. In various embodiments of the present invention, the MCU 404 measuring the status of each isolated pressure detection zones 206 could be resident within the Orthotic Insert 104, adjacent to it or remote from the insert.

FIG. 5 is a flow diagram illustrating the method for detection of pressure on the body surface, in accordance with an embodiment of the present invention. FIG. 5 illustrates a time based algorithm for detection of pressure 304 on the body surface, in accordance with an embodiment of the present invention. The time based algorithm would assist in detection of actuation pressure 304 events. If a pressure within any zone 206 exceeds the actuation pressure Pₘₐₓ, a timer, herein after referred to as actuation timer 502, for that event is started. The actuation timer 502 will continue to increment for a first time period, until either the first time period exceeds tₘₐₓ or until the pressure falls back below Pₘₐₓ. If it falls below Pₘₐₓ, a second timer, herein after referred to as relief timer 504, is started. The relief timer 504 measures a second time period since the pressure 304 in the zone 206 has remained under Pₘₐₓ. If the second time period exceeds a pre-determined time limit tₘᵢₙ the entire state for the zone is restarted. If the pressure rises above Pₘₐₓ before t′ₘᵢₙ expires, actuation timer 502 resumes. If the first time period exceeds tₘₐₓ an alert is communicated to the user to notify about actuation pressure detection. The alert 506 is communicated by a communication device upon receiving a signal from the computational device.

In accordance with an embodiment of the present invention, the body surface pressure detection system 600 can be comprised of a sensing pad 602 having the plurality of isolated pressure detection zones 206, a computational device 404 individually monitoring the zones 206 and a communication device 610 to report actuation pressure detection events using an alert indicator 606. In an embodiment, the sensing pad 602 can be a multi-layered pad made of flexible materials, in form of an Orthotic Insert 104. In various embodiments, the components of the body surface pressure detection system can be resident in the same Orthotic Insert 104 or can be physically separated. Further, power source for providing power to the system can be resident in the Orthotic Insert 104 or external.

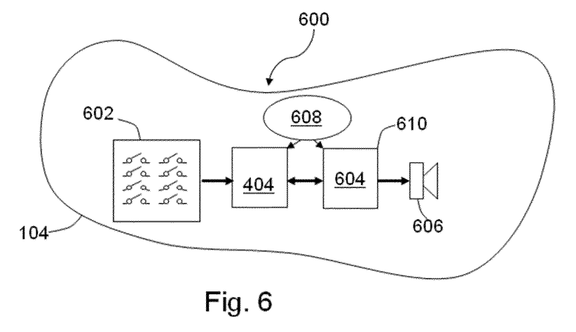

FIG. 6 is a diagram showing an exemplary arrangement of the components of the body surface pressure detection system with an integrated alert means, in accordance with an embodiment of the present invention. FIG. 6 shows logically how these subsystems would interconnect if realized in an Orthotic Insert 104 with an integrated alert means and power source. In this figure the sensing pad 602 is monitored by an MCU 404. An algorithm executed by the MCU 404 detects independent warning conditions in any or all of the zones 206 of the pad 602. When an alert condition is triggered, the MCU 404 drives a warning means 604. Such means can include visual indication through an LED or audible indication through an audio transducer such as a speaker or piezo-electric element. Power for the system can be derived from an integrated coin cell 608 battery.

One of the intrinsic benefits to using a sensing pad 602 designed to work with simple GPIO is that the MCU 404 can be held in a deep power down state most of the time. In this state all MCU 404 clocks and activity can be held static until an actuation pressure detection event is encountered on the associated MCU 404 GPIO pin. The MCU 404 can then use the event to trigger a power up interrupt whereby it can begin a suitable time based detection algorithm to determine if the event warrants an alarm indication. If that algorithm expires and no other pressure actuated GPIO event is triggered, the MCU 404 can return to a deep power down state and wait for the next event to occur.

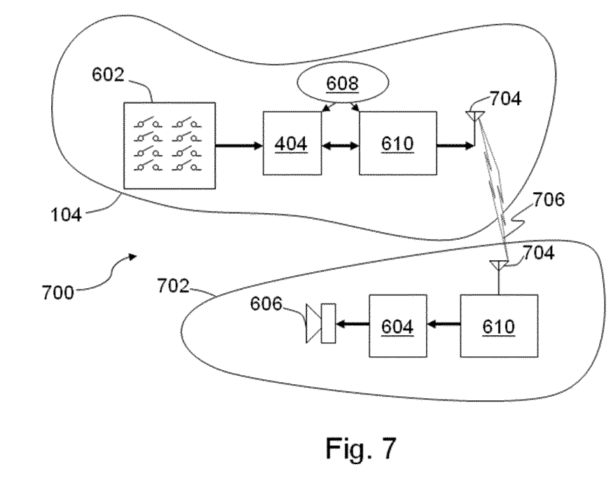

FIG. 7 is a diagram showing an exemplary arrangement of the components of the body surface pressure detection system with a wireless alert indication means, in accordance with an embodiment of the present invention. FIG. 7 shows logically how the subsystems would interconnect if realized in the body of an Orthotic Insert 104 with an internal power source but an external alert means, in accordance with another embodiment of the present invention. In this figure the sensing pad 602 is monitored by an MCU 404. An algorithm executed by the MCU 404 detects independent warning conditions in any or all of the zones of the pad. When an alert condition is triggered, the MCU 404 drives a radio transceiver 610 which communicates the alert condition to an external device such as a charm 702 that has a reciprocal transceiver 610. The charm 702 then provides an alert output by means that could include visual indication through an LED or audible indication through an audio transducer such as a speaker or piezo element. Power for the Orthotic Insert 104 can be derived from an integrated coin cell 608 battery. Power for the external charm 702 can be integrated as in the orthotic or external to both devices.

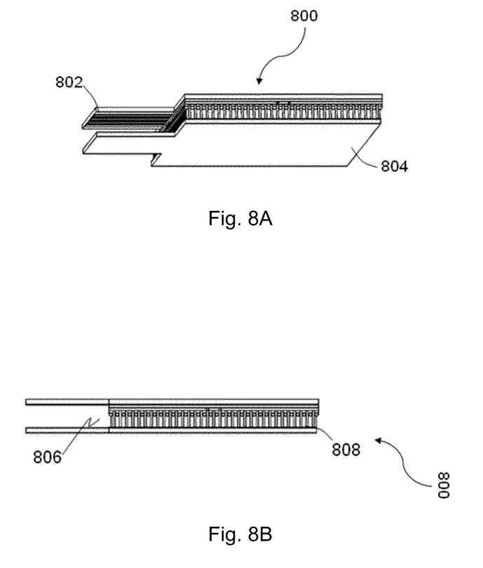

FIG. 8A is a diagram showing an exemplary arrangement of a fabric layer having printed conductors and the homogenously conductive layer, in accordance with an embodiment of the present invention. The sensing pad 602 can be comprised of multiple layers of flexible materials as illustrated in FIG. 8A, in accordance with an embodiment of the present invention. In this embodiment a top layer of isolative fabric having conductors 802 printed on the fabric is used to setup circuit paths with a homogeneously conductive fabric 804 at the bottom, touching the surface of the insole 102 of a shoe.

FIG. 8B is a magnified side-view of a multi-layered mat of flexible materials included in the body surface pressure detection system, in accordance with an embodiment of the present invention. In this embodiment conductive rubber nubs 806 are in contact with the compression spacer 808 made of foam. The foam compression spacer 808 is adhered to homogeneously conductive fabric layer 804 by the selective non-conductive adhesive 910.

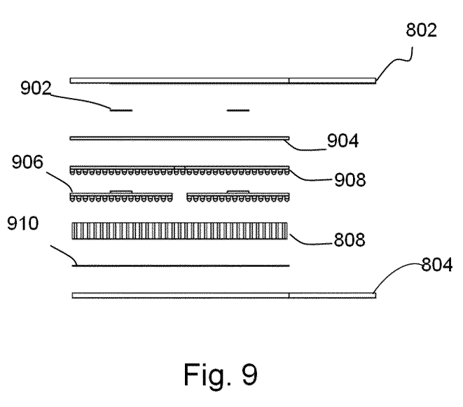

FIG. 9 is a side exploded view of the multi-layered pad, in accordance with an embodiment of the present invention. FIG. 9 illustrates a side exploded view of the sensing pad 602 in form of the multi-layered pad, showing details of one layer stack, in accordance with an embodiment of the present invention. The isolative fabric having the printed conductors 802 would be adhered to conductive rubber pads 906 having conductive nubs 806 as a physical feature on the opposing surface. This adhesive 902 would be electrically conductive and flexible as to allow for no perceptible change in flexibility at the point of adhesion. The rubber nubs 806 then become electrically common with their respective circuit trace printed on the top fabric layer.

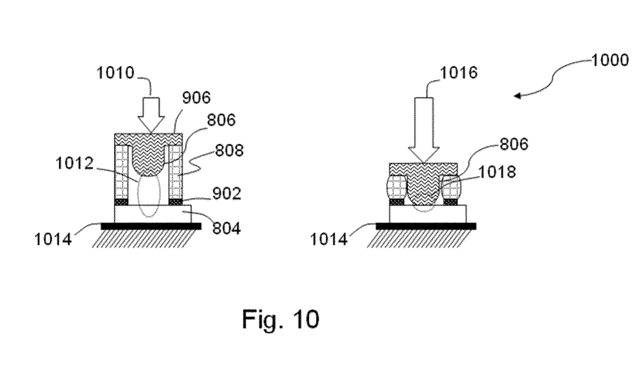

FIG. 10 is a view of a single column of a foam compression spacer with a single nub of a conductive rubber pad, in accordance with an embodiment of the present invention. FIG. 10 illustrates a view of a single column of a foam compression spacer 808 with a single nub 806 of a conductive rubber pad 906, in accordance with an embodiment of the present invention. As can be seen from the FIG. 10, the conductive rubber pad 906 transitions from an open electrical circuit 1012 to a closed electrical circuit 1018 when the nub 806 is compressed against a ridged base 1014, under actuation pressure.

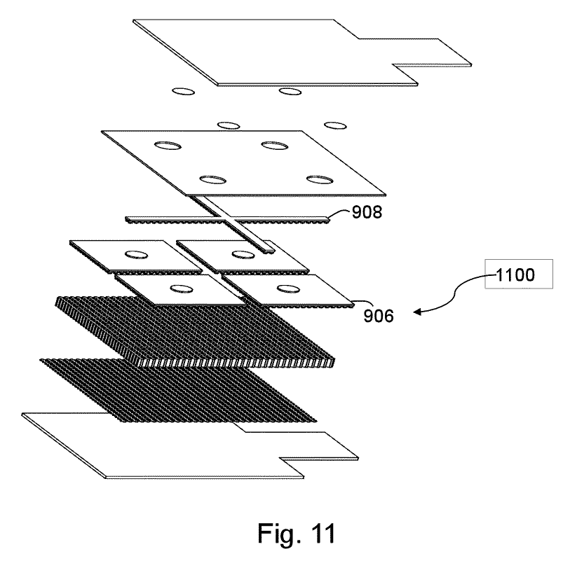

FIG. 11 is a perspective exploded view of the multi-layered pad, in accordance with an embodiment of the present invention. FIG. 11 shows an angled view of the features shown in FIG. 9. An important feature better shown by this angled view is the lateral insulator 908. The lateral insulator 908 assures electrical isolation of the four conductive rubber pads 906 shown in the FIG. 11. The insulator 908 is similar to the physical construction of the rubber pads 906 and is placed in such a manner that the physical character of the planar interface presented to the top surfaces of the sensing pad 602 appears and feels homogeneous. This feature characteristic is important in preventing the introduction of disjointed surfaces that can exacerbate or potentially create harmful pressure 304 gradients on the body surface.

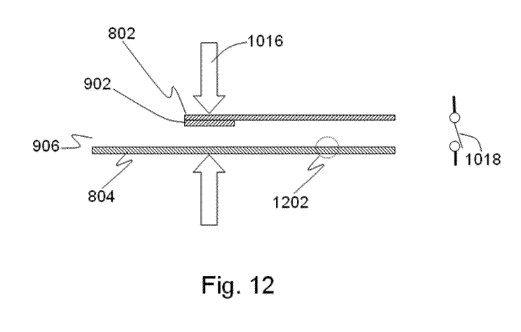

FIG. 12 is a diagram illustrating the formation of electrical connections on the multi-layered pad, in accordance with an embodiment of the present invention.pad FIG. 12 shows the electrically active parts of the layer stack that are in physical contact and become a closed circuit 1018 within a particular zone 206 when a pressure 304 greater than or equal to the actuation pressure is applied. The nubs 806 on the affected conductive rubber pad 906 make physical contact with the homogeneously conductive fabric 804 by compressing the foam compression spacer 808 sufficiently to do so. The selective adhesive 910 used to adhere the foam compression spacer 808 to the homogeneously conductive fabric 804 is selectively arranged as not to interfere with this contact action.

Pressure 304 gradients that actuate only a single nub 806 or small number of nubs 806 on a pad 602 will still register as a circuit closure. This feature increases the detection resolution of the pad 602 allowing it to detect gradients in areas much smaller than the pad itself.

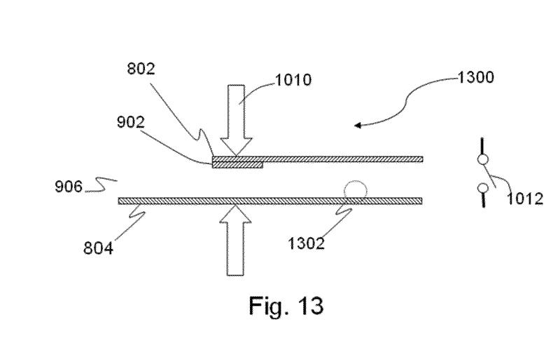

FIG. 13 is a diagram illustrating the breaking of electrical connections on the multi-layered pad, in accordance with an embodiment of the present invention.pad FIG. 13 shows the same electrically active parts of the layer stack when they are not subjected to a force greater than threshold 1016 or equal to the minimum threshold force. In this case they are physically separated by the counteracting reciprocal force of the foam compression spacer 808 and hence create an open circuit 1012.

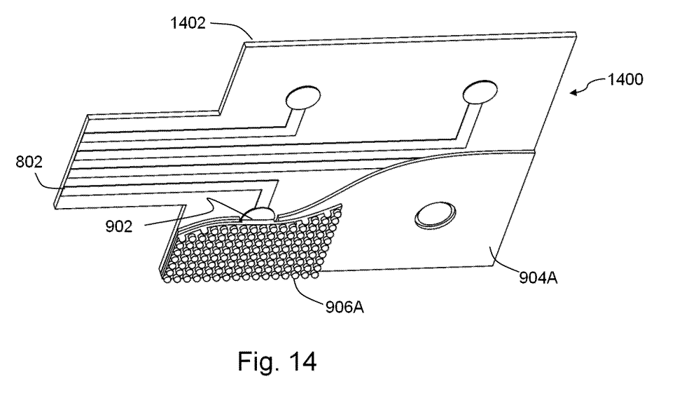

FIG. 14 is a perspective view of a cutaway of the multi-layered pad, in accordance with an embodiment of the present invention. FIG. 14 illustrates a cutaway of the multi-layered pad, depicting the manner in which the conductive rubber pads 906 within a zone 206 are attached to traces on the top fabric surface, in accordance with an embodiment of the present invention. The fabric insulator 904 prevents shorting between printed conductors 802 on the top fabric surface by way of electrical commons caused by contact with other conductive rubber pads 906 that are not part of the intended circuit.

The lateral insulator 908 is constructed of a material that is similar to the conductive rubber pads 906 in its physical characteristics including elasticity, but is non conductive. The shape and placement of the lateral insulator 908 is such that the texture and appearance of the top fabric layer is substantially homogeneous in both appearance and touch even under mechanical load.

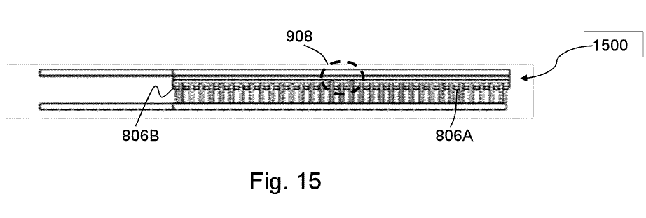

FIG. 15 is a side view of a multi-layered pad illustrating the placement of a lateral insulator between two electrically isolated conductive rubber contacts, in accordance with an embodiment of the present invention. FIG. 15 illustrates placement of a lateral insulator 908 between two electrically isolated conductive rubber contacts A 806A and conductive rubber contacts B 806B, in accordance with an embodiment of the present invention. The lateral insulator 906 prevents conductive rubber pads 906 within designated zones from shorting in a lateral direction with pads 906 in other zones by way of pad edges that are also electrically conductive. This insulator 906 is not necessary if the spacing between conductive rubber pads 906 is sufficient to prevent shorting when the rubber pads 906 are expanded under pressure or subjected to shear forces.

The invention has been described using example of an Orthotic Insert. However, a person skilled in the art can easily understand that the described body surface pressure detection system can be used for various purposes such as, gaming peripherals such as gloves or shoe sole inserts that provide input from on-body zones to entertainment devices. An example would be a shoe insert that sensed zones on the foot that would be used to synchronize dance steps with a game such as Dance Dance Revolution by Konami. Further, the multi-layered pad of the system can be constructed to take up shape of various body parts. Therefore, objects and embodiments of the invention should be construed according to the claims that follow below.

While the principles of the disclosure have been illustrated in relation to the exemplary embodiments shown herein, the principles of the disclosure are not limited thereto and include any modification, variation or permutation thereof.

What is claimed is:

1. A system for detecting pressure, the system comprising:
a multi-layered pad configured to contact a surface of a body; and
a plurality of pressure detection zones located on the multi-layered pad, each plurality of pressure detection zones are configured to detect a pressure exerted onto the multi-layered pad.

2. The system according to claim 1, further comprising:
a computational device configured to monitor the plurality of pressure detection zones; and
a communication device coupled to the computational device and configured to provide an indication upon receiving a signal from the computational device.

3. The system according to claim 1, wherein each of the plurality of pressure detection zones is physically and electrically isolated from one another.

4. The system according to claim 1, wherein the system further comprises a power source coupled to the multi-layered pad.

5. The system according to claim 1, wherein the multi-layered pad comprises more than two layers.

6. The system according to claim 1, wherein each of the plurality of pressure detection zones comprises a digital switch, each digital switch configured to activate upon an application of pressure above a pre-determined limit Pₘₐₓ to its corresponding pressure detection zone.

7. The system according to claim 2, wherein the computational device further comprises a micro-controller unit, the micro-controller unit detecting activation of the digital switch, the digital switch activating upon detecting a pressure above Pₘₐₓ corresponding to its pressure detection zone.

8. The system according to claim 7, the computational device further comprises an actuation timer, the actuation timer calculating a first time period corresponding to a pressure detection zone for which a pressure above Pₘₐₓ is detected.

9. The system according to claim 8, wherein the computational device sends the signal to the communication device when the pressure applied on a pressure detection zone remains above Pₘₐₓ for the first time period beyond a pre-determined time limit tₘₐₓ.

10. The system according to claim 2, wherein the computational device further comprises a relief timer, the relief timer starting when the pressure exerted on a pressure detection zone is greater than Pₘₐₓ but falls below Pₘₐₓ before the first time period reaches tₘₐₓ, and the relief timer calculating a second time period for which the pressure exerted on the pressure detection zone is less than Pₘₐₓ.

11. The system according to claim 10, wherein the relief timer for the pressure detection is reset when the second time period for the pressure detection zone is greater than a pre-determined time limit t′ₘᵢₙ.

12. The system according to claim 10, wherein the relief timer for the pressure detection zone is reset, and the actuation timer for the pressure detection zone is started when the second time period for the pressure detection zone is less than t′ₘᵢₙ and the pressure exerted on the pressure detection zone rises above Pₘₐₓ.

13. A system for detecting pressure, the system comprising:
a pad configured to contact a surface of a body;
a plurality of pressure detection zones located on the pad, the plurality of pressure detection zones are configured to detect a pressure exerted by the body onto the pad;
a computational device configured to monitor the plurality of pressure detection zones; and
a communication device coupled to the computational device and configured to provide an indication upon receiving a signal from the computational device.

14. The system according to claim 13, wherein each of the plurality of pressure detection zones is physically and electrically isolated from one another, and comprises a digital switch, each digital switch configured to activate upon an application of pressure above a pre-defined limit Pₘₐₓ to its corresponding pressure detection zone.

15. The system according to claim 13, wherein the computational device comprises a micro-controller unit, the micro-controller unit detecting activation of the digital switch, the digital switch activating upon detecting a pressure above Pₘₐₓ corresponding to its pressure detection zone.

16. The system according to claim 13, wherein the computational device further comprises:
an actuation timer, the actuation timer calculating a first time period corresponding to a pressure detection zone for which a pressure above Pₘₐₓ is detected and sending the signal to the communication device when the pressure applied on the pressure detection zone remains above Pₘₐₓ for the first time period beyond a pre-determined time limit tₘₐₓ; and
a relief timer, the relief timer starting when the pressure exerted on a pressure detection zone is greater than Pₘₐₓ but falls below Pₘₐₓ before the first time period reaches tₘₐₓ, and the relief timer calculating a second time period for which the pressure exerted on the pressure detection zone is less than Pₘₐₓ.

17. The system according to claim 13, wherein the pad comprises:
a layer of isolative fabric having a plurality of conductive traces printed on it; and
a plurality of conductive rubber pads attached to the plurality of conductive traces using a conductive adhesive.

18. The system according to claim 13, wherein the pad further comprises a layer of fabric insulator.

19. The system according to claim 13, wherein the pad further comprises a layer of homogenously conductive fabric.

20. The system according to claim 13, wherein the pad further comprises a lateral insulator layer.

21. The pad according to claim 17, wherein the adhesive is made of a flexible material and allows now perceptible change in the flexibility of the adhesive at the point of adhesion.

22. The pad according to claim 17, wherein the adhesive allows no perceptible change in flexibility.

23. The pad according to claim 17, wherein said conductive traces printed on the pad providing a plurality of circuit paths.

24. A system for detecting pressure, the system comprising:
a multi-layered pad configured to contact a surface of a body;
a plurality of physically and electrically isolated pressure detection zones located on the multi-layered mat, the plurality of pressure detection zones are configured to detect a pressure above a pre-defined limit Pₘₐₓ exerted by the body onto the multi-layered pad;
a computational device configured to monitor the plurality of pressure detection zones, the computational device comprises a micro-controller unit, the micro-controller unit detecting activation of a digital switch corresponding to a pressure detection zone, the digital switch activating on detecting the pressure to its corresponding pressure detection zone; and
a communication device coupled to the computation device and configured to provide an indication upon receiving a signal from the computational device.

25. The system according to claim 24, wherein the computational device comprises
an actuation timer, the actuation timer calculating a first time period for which a pressure above Pₘₐₓ is detected and sending the signal to the communication device when the first time period is greater than a pre-determined time limit tₘₐₓ; and
a relief timer, the relief timer calculating a second time period for which a pressure below a pre-defined limit Pₘₐₓ is detected.

26. The system according to claim 24, wherein the multi-layered mat comprises
a first layer of isolative fabric having a plurality of conductive traces printed on its first surface;
a plurality of conductive rubber pads attached to the plurality of conductive traces using a conductive adhesive;
a second layer of fabric insulator located between the plurality of conductive traces and the plurality of conductive rubber pads to prevent electrical shorting;
a third layer of homogenously conductive fabric maintained at a distance from the plurality of conductive rubber pads using a plurality of foam compression spacers, the plurality of foam compression spacers adhered to a second surface of the third layer facing the first surface using an adhesive; and
a fourth layer of lateral insulator located among the plurality of conductive rubber pads to prevent shorting in a lateral direction.

27. The system of claim 24, wherein the computational device is in a deep power down state until a pressure actuated event occurs on the at least one general purpose input output lines.

28. The system of claim 24, wherein the communication device comprises radio transceiver transmitting alert indication to the alert indicator.

29. The system of claim 24, wherein the alert indicator is a charm receiving communication signals from the radio transceiver.

30. The system of claim 24, wherein the source of the power comprises a coin cell coupled to, the computational device, the communication device, and the alert indicator.

31. The system of the claim 24, wherein the pad, the computational device, the communication device are integrated in the device, and wherein the alert indicator is integrated in the device or externally.
